(19) United States
(12) Patent Application Publication
(10) Pub. No.: US 2024/0423662 A1
(43) Pub. Date: Dec. 26, 2024

(54) SURGICAL ROUND KNIFE WITH ADJUSTABLE BLADE

(71) Applicant: Alcon Inc., Fribourg (CH)

(72) Inventors: Venjamin Stojanov, Dietlikon (CH); Niccolo Maschio, Winterthur (CH)

(21) Appl. No.: 18/738,244

(22) Filed: Jun. 10, 2024

Related U.S. Application Data
(60) Provisional application No. 63/509,580, filed on Jun. 22, 2023.

(51) Int. Cl. A61B 17/3211; A61B 17/00; A61B 17/34

(52) U.S. Cl. CPC A61B 17/3211; A61B 2017/0069; A61B 2017/00787; A61B 2017/347; A61B 2217/005

(57) ABSTRACT

A surgical knife for otologic procedures includes a handle comprising a distal end and a proximal end, the handle further comprising a control member and a shaft comprising a distal end and a proximal end, the proximal end coupled to the distal end of the handle. The surgical knife further comprises a rod disposed within the shaft and comprising a distal end and a proximal end, wherein the proximal end of the rod is coupled to the control member, and wherein the rod is configured to rotate about a first rotation axis upon actuation of the control member. The surgical knife further comprises a round blade rotatably coupled to the distal end of the rod and configured to rotate about a second rotation axis, wherein the second rotation axis is disposed at a fixed, non-zero angle relative to the first rotation axis.

### BACKGROUND

The human ear is subject to a variety of disorders including, but not limited to, acoustic neuroma, acoustic trauma, balance disorders including vertigo, barotrauma, cholesteatoma, exostosis, hearing loss, labyrinthitis, Ménière's disease, ossicular chain dislocation, outer ear infections, middle ear infections, vestibular neuritis, vestibular schwannomaotosclerosis, schwannoma, tinnitus, and tympanic membrane perforations, to provide a few examples.

Access to the ear canal, tympanic membrane, middle ear, and inner ear has historically been challenging due to angulation and/or small size of such structures, the delicateness of various ear tissues, and the relatively large size of available surgical instruments. Moreover, instruments specifically designed for various otologic (ear) procedures are lacking.

Therefore, there is a need for improved methods, devices, and surgical instruments for otologic procedures.

### SUMMARY

Aspects of the present disclosure relate to instruments for otologic procedures, and more particularly, to cutting instruments for otologic surgical procedures.

In certain embodiments, a surgical knife for otologic procedures is provided. The surgical knife comprises a handle comprising a distal end and a proximal end opposite the distal end, the handle further comprising a control member, and a shaft comprising a distal end and a proximal end opposite the distal end, the proximal end coupled to the distal end of the handle. The surgical knife further comprises a rod disposed within the shaft and comprising a distal end and a proximal end opposite the distal end, wherein the proximal end of the rod is coupled to the control member, and wherein the rod is configured to rotate about a first rotation axis upon actuation of the control member, and a round blade rotatably coupled to the distal end of the rod and configured to rotate about a second rotation axis, wherein the second rotation axis is dispose at a fixed, non-zero angle relative to the first rotation axis.

### BRIEF DESCRIPTION OF THE DRAWINGS

The drawings described herein are for illustrative purposes only, are schematic in nature, and are intended to be exemplary rather than to limit the scope of the disclosure.

FIGS. 1A-1F illustrate various operations of an otologic procedure wherein a tympanomeatal flap is formed using a round knife and a separate straight knife.

FIGS. 2A-2B illustrate partial cross-sectional side views of a surgical knife having a rotatable round blade, according to embodiments described herein.

FIG. 2C illustrates a detailed perspective view of an exemplary universal joint for facilitating rotation of the round blade of FIGS. 2A-2B, according to embodiments described herein.

FIG. 2D illustrates a schematic side view of an exemplary bevel gear system for facilitating rotation of the round blade of FIGS. 2A-2B, according to embodiments described herein.

FIGS. 2E-2G illustrate detailed partial cross-sectional side views of exemplary locking mechanisms for the surgical knife of FIGS. 2A-2B, according to embodiments described herein

FIGS. 3A-3B illustrate schematic side views of another surgical knife having a round blade with an adjustable tilt angle, according to embodiments described herein.

FIGS. 3C-3D illustrate detailed cross-sectional side views of an exemplary fulcrum mechanism for facilitating adjustment of the angle of the round blade of FIGS. 3A-3B, according to embodiments described herein.

FIGS. 3E-3G illustrate detailed side views of an exemplary locking mechanism for the surgical knife of FIGS. 3A-3B, according to embodiments described herein.

FIG. 4 illustrates a detailed perspective view of a round blade in an unrotated position, according to embodiments described herein.

FIG. 5 illustrates a schematic side view of an exemplary surgical knife, in an unrotated position, according to embodiments described herein.

The above summary is not intended to represent every possible embodiment or every aspect of the subject disclosure. Rather, the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to instruments for otologic (car) procedures, and more particularly, to cutting instruments for otologic surgical procedures.

A surgeon may use several surgical instruments for the successful completion of an otologic surgical procedure. Two of such instruments include a round knife and a straight knife. For example, during middle ear surgeries in which the tiny, delicate structures within or adjacent to the middle ear cavity are operated on, a tympanomcatal flap may be formed in the external auditory canal (EAC) using both a round knife and a straight knife. Together with the tympanic annulus, the tympanomcatal flap may be elevated to provide a surgeon with access to the middle ear space. Examples of procedures during which a tympanomcatal flap may be formed include tympanoplastics, ossiculoplastics, and stapes surgeries, as well as other types of postauricular, transcanal, or endural middle ear surgical procedures, to name a few.

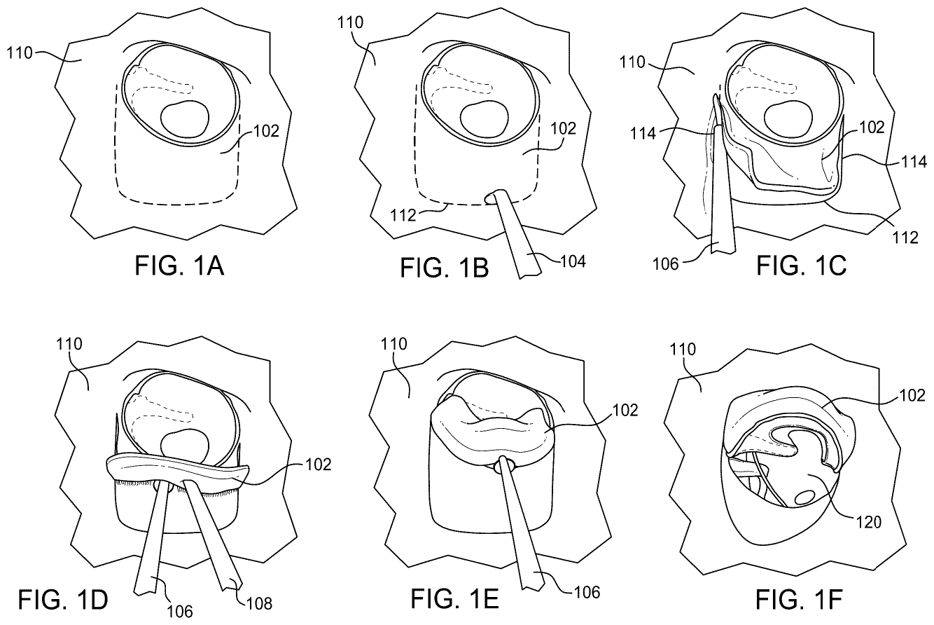

FIGS. 1A-1F illustrate stepwise operations in the formation of a tympanomcatal flap 102 during a postauricular procedure utilizing a round knife and a separate straight knife. Generally, a series of incisions are made at various angles to create the tympanomcatal flap 102, which may thereafter be lifted/folded to gain access to the anatomy beneath. FIG. 1A illustrates an incision outline of the tympanomeatal flap 102 to be cut from tympanomeatal tissue 110. In FIG. 1B, a round knife 104 is utilized to create a first, “horizontal” incision 112 that is substantially perpendicular to the longitudinal axis of the EAC. In certain examples, the round knife 104 may be used to “crush” the tympanomeatal tissue 110 to reduce bleeding and maintain a smoothly cut incision 112. Thereafter, in FIG. 1C, a straight knife 106 is utilized to create two “vertical” incisions 114 that are substantially parallel to the longitudinal axis of the EAC and intersect with the incision 112. The combination of the two “vertical” incisions 114 and the “horizontal” incision 112 form the tympanomeatal flap 102. In some certain examples, prior to forming the incisions 114, the surgeon may “tunnel” superiorly under the tympanomeatal tissue 110 where the incisions 114 are to be made to facilitate smooth cutting. In FIGS. 1D-1E, the round knife 104 and a separate auxiliary tool, such as a suction tube 108, may be utilized to manually elevate/fold the tympanomeatal flap 102 in the EAC toward the annulus. Finally, in FIG. 1F, the annulus along with the tympanomeatal flap 102 is elevated and the middle ear space 120 is exposed to provide the surgeon access to the middle ear space 120.

The utilization of multiple different instruments during an otologic procedure, such as during the formation of the tympanomeatal flap 102 in FIGS. 1A-1F, and their repeated insertion and/or removal into the ear may increase the risk of unwanted trauma to surrounding ear tissues, and/or increase the time needed for surgery. Further, the repeated exchange of instruments may decrease the efficiency of the otologic procedure and increase inconvenience for the surgeon.

To address some of the aforementioned concerns, the present disclosure provides an otologic surgical knife having an adjustable round blade to facilitate the formation of different types of incisions with a single instrument, thereby reducing the number of instruments needed during certain otologic procedures. Such a surgical knife may be utilized, for example, to create and elevate/fold a tympanomeatal flap in the external auditory canal for access to the middle ear space. Because the surgical knife comprises an adjustable round blade, a surgeon may maintain and utilize the same knife to make various types of incisions in the external auditory canal. Thus, in certain examples, the surgical knife described herein reduces the number of instrument exchanges during an otologic procedure, which in turn increases the case of use and convenience for the surgeon, improves procedural efficiency, reduces the overall time for surgery, and reduces the risk of trauma to the patient's ear tissues.

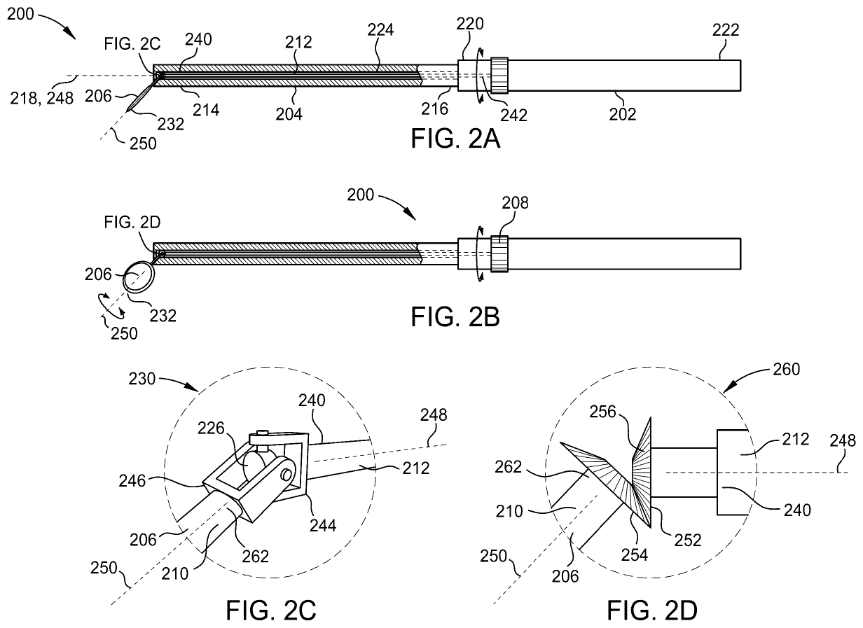

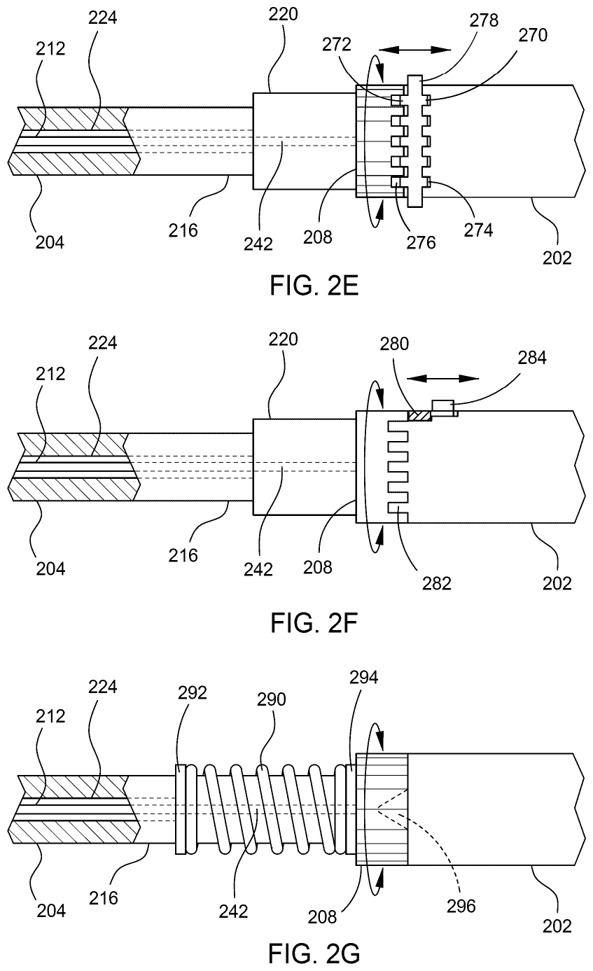

FIGS. 2A-2B illustrate partial cross-sectional side views of an exemplary surgical knife 200, according to certain embodiments described herein. As shown, the surgical knife 200 generally includes a handle 202 and a shaft 204 coupled to and extending distally from a distal end 220 of the handle 202. In certain embodiments, the shaft 204 is removably coupled to the handle 202. In certain embodiments, the shaft 204 is fixedly coupled to the handle 202.

The handle 202 further includes a proximal end 222 opposite the distal end 220. In certain embodiments, handle 202 is a hand piece having an outer surface configured to be held by a user, e.g., a surgeon. As such, the outer surface of the handle 202 may be ergonomically contoured for holding by the user. In certain embodiments, the outer surface of the handle 202 may be textured or have one or more gripping features formed thereon, such as one or more grooves, ridges, and/or other patterns. Generally, the handle 202 may be made from any material(s) commonly used for such instruments and suitable for otologic surgery. For example, the handle 202 may be formed of lightweight aluminum, stainless steel (or other metal alloys), a thermoplastic polymer, and/or other suitable material(s). In certain embodiments, the handle 202 may be configured to be sterilized and used in more than one surgical procedure. In other embodiments, the handle 202 is configured for a single use.

The shaft 204 generally comprises an elongated tubular member having a proximal end 216 coupled to the handle 202 and a distal end 214 opposite the proximal end 216. In certain embodiments, the shaft 204 has a cylindrical shape. In certain embodiments, the shaft 204 has a polygonal tubular shape. In certain embodiments, the shaft 204 is substantially linear. In certain embodiments, the shaft 204 comprises a curvature. However, any suitable shapes and/or morphologies are contemplated for the shaft 204.

Similar to the handle 202, the shaft 204 may be made from any material(s) commonly used for such instruments and suitable for otologic surgery. For example, the shaft 204 may be formed of lightweight aluminum, stainless steel (or other metal alloys), a thermoplastic polymer, and/or other suitable material(s). In certain embodiments, the shaft 204 is made from the same material as the handle 202. In certain embodiments, the shaft 204 is made from a different material than the handle 202. In certain embodiments, the shaft 204 may be configured to be sterilized and used in more than one surgical procedure. In other embodiments, the shaft 204 is configured for a single use.

In certain embodiments, a major axis 218 of the shaft 204 is parallel or collinear with a major axis of the handle. However, in other embodiments, to improve visibility for the surgeon, the major axis 218 of the shaft 204 may be disposed at an angle relative to the major axis of the handle 202. In certain embodiments, the shaft 204 may be coupled to the handle 202 at an angle between about 0 degrees and about 90 degrees relative to the major axis 218 of the shaft 204 and/or the major axis of the handle 202. For example, in certain embodiments, the shaft 204 may be coupled to the handle 202 at an angle between about 30 degrees and about 60 degrees relative to the major axis of the handle 202, such as an angle of about 45 degrees. Further, in certain embodiments, the shaft 204 may be rotatable relative to the handle 202. In certain embodiments, the shaft 204 may have a maximum diameter between about 1 millimeter to about 3 millimeters to further improve visibility and prevent the shaft 204 from blocking the surgeon's view of, e.g., the middle ear space during a procedure.

As shown in FIGS. 2A-2B, a channel 224 extends from the handle 202 to the distal end 214 of the shaft 204, or from the handle 202 to substantially near the distal end 214, and comprises a rod 212 disposed therein. The rod 212 is configured to rotate about a primary axis of rotation 248, which may be collinear and/or parallel with a major longitudinal axis of the rod 212 and, in certain embodiments as shown in FIG. 2A, the major axis 218 of the shaft 204. A distal end 240 of the rod 212 is operably coupled to a round blade 206 within the shaft 204 via any suitable type of joint and/or components. Meanwhile, a proximal end 242 of the rod 212 is operably coupled to a control member 208 within the handle 202. Actuation of the control member 208 by a user causes the rod 212 to rotate around the primary axis of rotation 248, which in turn causes the round blade 206 operably coupled therewith to rotate around a secondary axis of rotation 250.

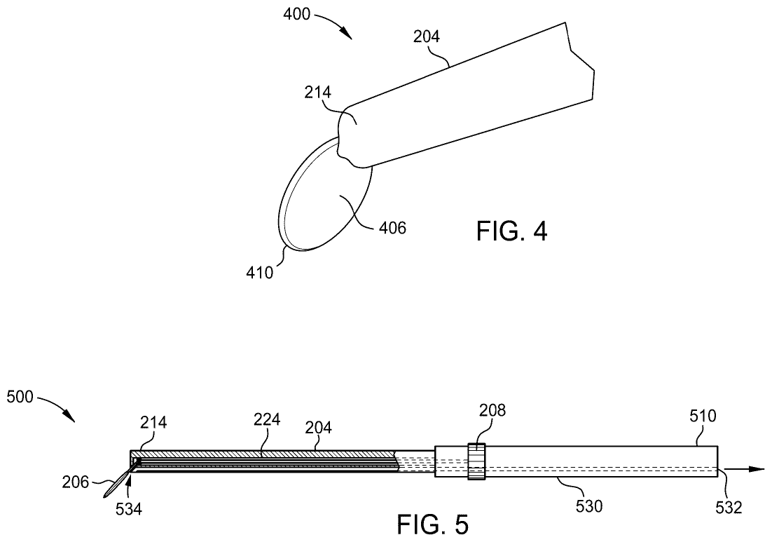

The round blade 206 is rotatably coupled to the distal end 214 of the shaft 204 and rotates about the secondary axis of rotation 250. In certain embodiments, the round blade 206 is coupled to the distal end 214 such that the secondary axis of rotation 250 of the round blade 206 is disposed at a fixed, non-zero angle between about 30 degrees to about 70 degrees relative to the major axis 218 of the shaft 204 and/or primary axis of rotation 248, such as an angle between about 40 degrees to about 60 degrees relative to the major axis 218 and/or primary axis of rotation 248. In certain embodiments, the round blade 206 comprises a disc-like blade with a rounded cutting edge 232; however, any suitable morphologies for the round blade 206 are contemplated. Further details regarding the morphology of the round blade 206 are illustrated and described with reference to FIG. 4.

In certain embodiments, the control member 208 may be manually adjusted by the user between at least a first position to a second position, wherein adjustment of the control member 208 between the first position and the second position causes the round blade 206 to rotate about the secondary axis of rotation 250 at a rotational angle between about 0 degrees and about 90 degrees. For example, in certain embodiments, adjustment of the control member 208 between the first position and the second position causes the round blade 206 to rotate about the secondary axis of rotation 250 at a rotational angle between about 10 degrees and about 80 degrees, such as a rotational angle between about 20 degrees and about 70 degrees, such as a rotational angle between about 30 degrees and about 60 degrees, such as a rotational angle between about 40 degrees and about 50 degrees, such as a rotational angle of about 45 degrees. In certain embodiments, adjustment of the control member 208 between the first position and the second position causes the round blade 206 to rotate about the secondary axis of rotation 250 at a rotational angle between about 0 degrees and about 360 degrees, such as a rotational angle between about 0 degrees and about 180 degrees.

In certain embodiments, when the control member 208 is disposed in the first position, the cutting edge 232 round blade 206 may be optimally disposed at an angle/orientation to facilitate the formation of a horizontal incision for creating a tympanomcatal flap, as described above in FIG. 1B, or to facilitate lifting of the tympanomeatal flap to expose the middle ear space, as described above in FIGS. 1D-1E. In certain embodiments, when the control member 208 is disposed in the second position, the cutting edge 232 round blade 206 may be optimally disposed at an angle/orientation to facilitate formation of the two vertical incisions for creating the tympanomeatal flap, as described above in FIG. 1C.

In certain embodiments, the control member 208 comprises a rotating component, such as a rotating knob, configured to rotate around the handle 202 upon manual actuation by the user, as shown in FIG. 2A. In such embodiments, rotation of the control member 208 may directly or indirectly cause rotation of the rod 212, and thus, the blade 206. However, any suitable types of actuation mechanisms and/or components for controllably rotating the rod 212 and blade 206 are further contemplated, such as a sliding button driving a leadscrew, a squeezing mechanism, or a button controlling a small electromotor configured to rotate the rod 212.

In certain embodiments, the control member 208 may further include a locking mechanism. For example, the locking mechanism may comprise a rotating multi-groove system or a friction-based system, as described with reference to FIGS. 2E-2G.

In such embodiments, when the locking mechanism of the control member 208 is engaged, any rotation of the rod 212 and/or blade 206 may be prevented. Accordingly, prior to making a desired type of incision, the user (e.g., a surgeon) may actuate the control member 208 until the round blade 206 is rotated to a rotational position optimal for facilitating the desired incision type and may then lock the control member 208 in place. In certain embodiments, once the control member 208 is locked, the round blade 206 will remain fixed in position and/or orientation until the locking mechanism is released. Such locking functionality reduces the likelihood of undesired movement of the round blade 206 during an otologic procedure, thereby improving the safety and efficiency thereof.

FIG. 2C illustrates a detailed perspective view of an exemplary universal joint 230 for coupling the rod 212 to the round blade 206, according to embodiments described herein. As described above, the rod 212 extends along and is configured to rotate about the primary axis of rotation 248, while the round blade 206 is configured to rotate about the secondary axis of rotation 250. Since the primary axis of rotation 248 and secondary axis of rotation 250 are noncollinear and nonparallel, the universal joint 230, also known as a U-joint, may be utilized to operably couple the rod 212 and round blade 206 (e.g., transfer mechanical rotational motion from the rod 212 to the round blade 206) while facilitating rotation of each component about its respective axis of rotation.

As shown, the universal joint 230 includes a first hinge 244 coupled to the distal end 240 of the rod 212, and second hinge 246 coupled to a proximal end 262 of a base 210 of the round blade 206. The first hinge 244 and the second hinge 246 are movably coupled to each other via a cross shaft 226, which enables the hinges 244, 246 to simultaneously rotate about the noncollinear and nonparallel axes of rotation 248, 250, respectively. Note that the universal joint 230 depicted in FIG. 2C is only exemplary, and other types and/or arrangements of universal-type joints are further contemplated.

FIG. 2D illustrates a detailed schematic side view of an exemplary bevel gear system 260 for coupling the rod 212 to the round blade 206, according to embodiments described herein. Similar to the universal joint 230, the bevel gear system 260 enables the transfer of mechanical rotational motion of the rod 212 to mechanical rotational motion of the round blade 206, despite the rod 212 and round blade 206 rotating about noncollinear and nonparallel axes.

As shown, the bevel gear system 260 includes a first bevel gear 252 fixedly coupled to the distal end 240 of the rod 212, and a second bevel gear 254 fixedly coupled to the proximal end 262 of the base 210 of the round blade 206. The first and second bevel gears 252, 254 may each comprise toothed rotating elements having a plurality of radially disposed teeth 256 that are mated or engaged with the teeth 256 of the other, such that rotation of one of the first bevel gear 252 and the second bevel gear 254 causes rotation of the other. In certain embodiments, the first bevel gear 252 may be mated with the second bevel gear 254 at a fixed angle corresponding to the angle formed between the primary axis of rotation 248 and the secondary axis of rotation 250.

FIGS. 2E-2G illustrate detailed partial cross-sectional side views of various locking mechanisms that, when engaged, prevent the control member 208 from rotating and therefore changing the position of the rod 212 and/or the blade 206 during use of the surgical knife 200.

Turning to FIG. 2E, a partial cross-sectional side view of a first rotating multi-groove locking system is illustrated. The multi-groove locking system in FIG. 2E includes a sliding ring 278, which has a plurality of annularly- or circumferentially-disposed protrusions on both distal and proximal sides thereof, referred to herein as distal protrusions 272 and proximal protrusions 270. The distal protrusions 272 may engage circumferentially with corresponding annularly- or circumferentially-disposed notches 276 formed in the control member 208 or another intermediary component disposed therebetween, while the proximal protrusions 270 may engage with corresponding annularly- or circumferentially-disposed notches 274 formed in the handle 202 or another intermediary component disposed therebetween. To rotate the control member 208 and adjust the round blade 206, the surgeon may slide the sliding ring 278 distally to unlock/disengage the proximal protrusions 270 from the notches 274 in the handle 202 while the distal protrusions 272 are kept engaged with the notches 276 of the control member 208. In this position, the surgeon may rotate the control member 208, thereby rotating the rod 212 and the blade 206 to a desired position. To lock the control member 208 and prevent rotation, the surgeon may slide the sliding ring 278 proximally to engage the proximal protrusions 270 with the notches 274 in the handle 202, while the distal protrusions 272 are kept engaged with the notches 276 of the control member 208. To facilitate constant engagement of the distal protrusions 272 with the notches 276 in both locked and unlocked positions, the distal protrusions 272 and/or the notches 276 may be appropriately sized (e.g., larger) relative to the protrusions 270 and/or notches 274. In some embodiments, the sliding ring 278 may also be proximally biased (e.g., using a spring mechanism) to maintain or transition the control member 208 in the locked position without requiring user adjustment.

In FIG. 2F, a partial cross-sectional view of a second rotating multi-groove locking system is illustrated. The locking system in FIG. 2F includes a key 280 that is attached to a sliding element 284 on the handle 202. The key 280, upon translation of the sliding element 284 by a surgeon, is configured to slidably engage or disengage with one of a plurality of annularly- or circumferentially-disposed notches 282 in control member 208, or an intermediary component disposed therebetween, to lock and unlock the control member 208, respectively. For example, the surgeon may slide the sliding element 284 proximally to disengage the key 280 from one of the plurality of notches 282 in the control member 208, allowing the surgeon to then rotate the control member 208, and thus the round blade 206, to a desired position. Once the round blade 206 is in a desired position, the surgeon may then slide the sliding element 284 distally to engage the key 280 with another one of the plurality of notches in the control member 208, thereby locking the control member 208 and preventing rotation of the round blade 206. In some embodiments, the sliding element 284, and thus the key 280, may be distally biased (e.g., using a spring mechanism) to maintain or transition the control member 208 in the locked position without requiring user adjustment.

Turning now to FIG. 2G, a partial cross-sectional view of a continuous friction-based locking mechanism is illustrated. The locking mechanism of FIG. 2G utilizes the frictional force, or resistance, between the control member 208 and the handle 202, and/or any intermediary components disposed therebetween, to maintain the control member 208 in a locked or secured position. In such embodiments, the locking mechanism may have continuous positioning, or be without fixed preset positions, such that the locking mechanism may be locked or maintained at any point or increment along a range of positions. To rotate the control member 208, the surgeon may apply a rotational force to the control member 208 greater than that of the friction force between the control member 208 and the handle 202, thereby allowing adjustment of the control member 208 and thus, the round blade 206. In certain embodiments, the friction force between the control member 208 and the handle 202 may be modified (e.g., increased) utilizing a coating or surface finishing (e.g., roughening, polishing, surface features) that facilitates increased friction between the handle 202 and the control member 208. In certain embodiments, the handle 202 may further have a conical portion 296 configured to engage with a corresponding surface of the control member 208 to increase the contact surface area between the control member 208 and handle 202 for increased friction.

In still further embodiments, the control member 208 may be biased against the handle 202 by a biasing member to increase frictional resistance when rotating the control member 208. For example, the locking mechanism in FIG. 2G includes a spring 290 disposed at a proximal end of the shaft 204. The spring 290 couples to a first mechanical stop 292 (e.g., a disc) disposed on or around the shaft 204 at a distal end of the spring 290 and a second mechanical stop 294 (e.g., another disc) coupled to the control member 208, or the control member 208 itself, at a proximal end of the spring 290. The spring 290 creates increased frictional force between the control member 208 and the handle 202 by proximally biasing the control member 208 against the handle 202. To rotate the control member 208, the surgeon may apply a rotational force to the control member 208 greater than that of the friction force between the control member 208 and the handle 202, thereby allowing adjustment of the control member 208. In certain embodiments, the surgeon may also distally push the control member 208 while rotating the control member 208, thereby compressing the spring 290 and eliminating the friction between the control member 208 and the handle 202 to allow the control member 208, and thus rod 212 and blade 206, to rotate more easily.

The ability to rotate the round blade 206 of surgical knife 200 facilitates safer and more efficient performance of otologic procedures. As an illustrative example, the surgical knife 200 may be used to efficiently form and elevate a tympanomcatal flap during a postauricular middle ear surgery, giving the surgeon access to the middle ear space. For example, a surgeon may first insert the surgical knife 200 into the external auditory canal (EAC) with the round blade 206 in a first rotational orientation or position that facilitates “horizontal” incisions. The surgeon may then use the round blade 206 to create a horizontal incision in the tympanomeatal tissue, as described in FIG. 1B above, that is substantially perpendicular to the longitudinal axis of the EAC. Thereafter, the surgeon may, via manual manipulation of the control member 208, rotate the round blade 206 to a second rotational orientation or position that facilitates “vertical” incisions. The surgeon may then use the rotated round blade 206 to create two vertical incisions in the tympanomeatal tissue that are substantially parallel to the longitudinal axis of the EAC, as described in FIG. 1C above. The two vertical incisions may be cut with the rotated round blade 206 such that they intersect with the prior horizontal incision. Thereafter, the surgeon may use the control member 208 to transition the round blade 206 back to the first orientation. The surgeon may then use the round blade 206 to lift the tympanomeatal flap formed by the horizontal and vertical incisions, thereby allowing the surgeon access to the middle ear space.

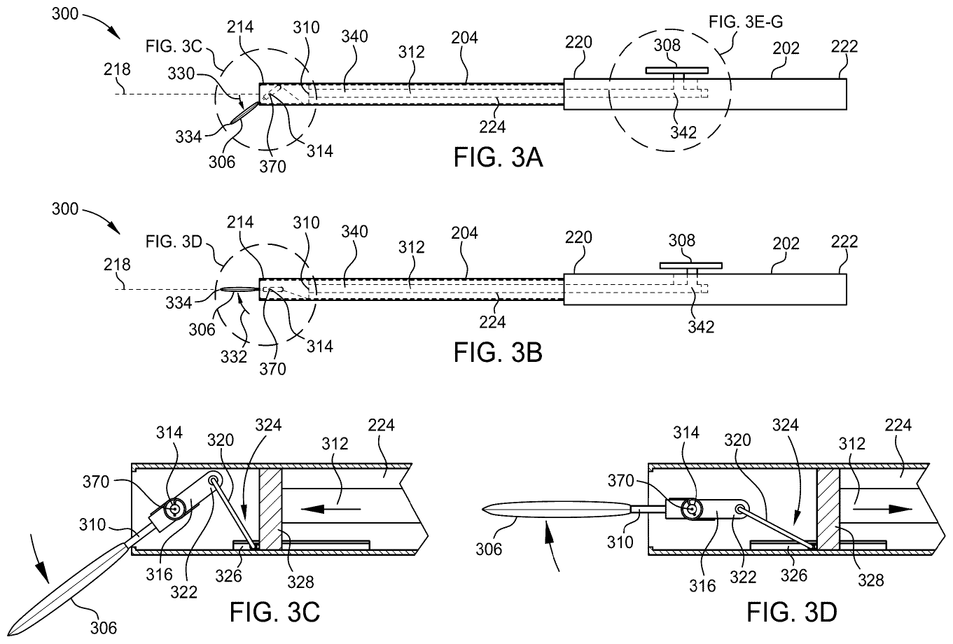

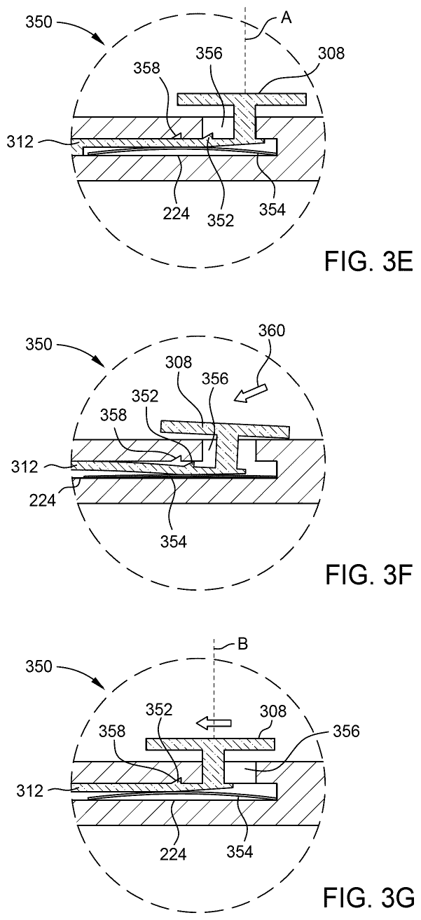

FIGS. 3A-3B illustrate schematic side views of another exemplary surgical knife 300, according to embodiments described herein. The surgical knife 300 is substantially similar to the surgical knife 200 and also comprises an adjustable round blade 306; however, instead of the round blade 306 being rotatably adjustable, the round blade 306 comprises an adjustable tilt angle. In other words, rather than being rotationally adjustable about a longitudinal axis of the round blade 306 (and/or a base 310 thereof), the round blade 306 may be tilted (e.g., rotated) about a lateral axis 370 perpendicular to the longitudinal axis of the round blade 306 and a major longitudinal axis 218 of the shaft of the surgical knife 300.

For clarity, features of the surgical knife 300 like or similar to those of surgical knife 200 are labelled with like or similar reference numerals and are omitted from the following description for brevity.

In FIGS. 3A-3B, a rod 312 is disposed within the channel 224 of the surgical knife 300 and extends substantially from the handle 202 to the distal end 214 of the shaft 204. The rod 312 is configured to translate longitudinally within the channel 224 along an axis that may be collinear and/or parallel with the major longitudinal axis 218 of the shaft 204. A distal end 340 of the rod 312 is operably coupled with the round blade 306 within the shaft 204 via any suitable type of joint and/or components to facilitate adjustment (e.g., rotation) of the round blade 306 about a rotational axis 370 perpendicular to the major axis 218 of the shaft 204. Meanwhile, a proximal end 342 of the rod 312 is operably coupled to a control member 308 within the handle 202. Actuation of the control member 308 by a user causes the rod 312 to laterally translate within the channel 224, which in turn causes the round blade 306 operably coupled therewith to rotate around the axis 370 perpendicular to the major axis 218 (e.g., axis 270 extends into and out of the page). Thus, actuation of the control member 308 causes adjustment of a “tilt” angle of the round blade 306 relative to the major axis 218 of the shaft 204, represented in FIGS. 3A-3B by arrows 330 and 332 respectively.

In certain embodiments, the control member 308 may be manually adjusted by the user between at least a first position to a second position, wherein adjustment of the control member 308 between the first position (FIG. 3A) and the second position (FIG. 3B) causes the round blade 306 to “tilt” or rotate about the axis 370 at a rotational angle between about 0 degrees and about 45 degrees, between about 0 degrees and about 60 degrees, or between about 0 degrees and 75 degrees. For example, in certain embodiments, adjustment of the control member 308 between the first position and the second position causes the round blade 306 to rotate about the axis 370 at a rotational angle between about 5 degrees and about 40 degrees, such as a rotational angle between about 10 degrees and about 35 degrees, such as a rotational angle between about 15 degrees and about 30 degrees, such as a rotational angle between about 20 degrees and about 25 degrees.

In certain embodiments, when the control member 308 is disposed in the first position, the cutting edge 334 of the round blade 306 may be optimally disposed at an angle/orientation to facilitate the formation of a horizontal incision for creating a tympanomeatal flap, as described above in FIG. 1B, or to facilitate lifting of the tympanomeatal flap to expose the middle ear space, as described above in FIGS. 1D-1E. In certain embodiments, when the control member 308 is disposed in the second position, the cutting edge 334 of the round blade 306 may be optimally disposed at an angle/orientation to facilitate formation of the two vertical incisions for creating the tympanomeatal flap, as described above in FIG. 1C.

In certain embodiments, the control member 308 comprises a sliding button or other sliding toggle. Accordingly, the control member 308 may be translated from a position nearest the proximal end 222 of the handle 202 to a position nearest the distal end 220 of the handle 202, and vice versa. In certain embodiments, the position nearest the distal end 220 of the handle 202 is the first position, and the position nearest the proximal end 222 of the handle 202 is the second position. In certain other embodiments, the position nearest the distal end 220 of the handle 202 is the second position, and the position nearest the proximal end 222 of the handle 202 is the first position. The longitudinal translation of the control member 308 causes translation of the rod 312 towards or away from the distal end 214 of the shaft 204, which in turn causes adjustment of the tilt angle of the round blade 306. Although shown and described as a sliding actuation mechanism, any suitable types of actuation mechanisms and/or components for controllably adjusting the tilt angle of the blade 306 are further contemplated, such as a rotating knob and the like.

In certain embodiments, the control member 308 may further include a locking mechanism. One example of a suitable locking mechanism is shown and described with reference to FIGS. 3E-3G below.

FIGS. 3C-3D illustrate detailed cross-sectional side views of an exemplary fulcrum mechanism for facilitating adjustment of the tilt angle of the round blade 306 of FIGS. 3A-3B, according to embodiments described herein. Note that the mechanism illustrated in FIGS. 3C-3D is only exemplary, and other suitable mechanisms and/or components for adjusting the tilt angle of the round blade 306 are further contemplated.

As shown, the round blade 306 is rotatable about a fulcrum 314, which comprises the rotational axis 370 perpendicular to the major axis 218 of the shaft 204. In the depicted embodiments, the round blade 306 rotatably couples to the fulcrum 314 at a base 316 of the round blade 306, which may be disposed at a proximal end of the round blade 306 and housed within the shaft 204. To controllably rotate the round blade 306 about the fulcrum 314, a proximal end 322 of the base 316, which is disposed proximal to the connection point of the base 316 with the fulcrum 314, is operably coupled to the distal end 340 of the rod 312. Thus, the round blade 306 is operably coupled, either directly or indirectly, to the distal end 340 of the rod 312 at the distal end 214 of the shaft 204.

In FIGS. 3C-3D, the base 316 of the round blade 306 is operably coupled to the rod 312 via a hinge arm 320 and sliding assembly 324. The hinge arm 320 is movably (e.g., rotatably) coupled to the base 316 and the sliding assembly 324 at opposing ends of the hinge arm 320. Meanwhile, the sliding assembly 324 is coupled to the distal end 340 of the rod 312 and comprises one or more components configured to transfer mechanical motion of the rod 312 onto the hinge arm 320 and thus, the base 312 of the round blade 306, for adjusting the tilt angle of the round blade 306. In the example of FIGS. 3C-3D, the sliding assembly includes a connector 328 and a slider 326. The connector 328 couples the distal end 340 of the rod 312 to the slider 326, which is coupled to the hinge arm 320 and is configured to longitudinally translate along, or adjacent to, a sidewall of the shaft 204.

During use, longitudinal translation of the rod 312 within the shaft 204 (e.g., via actuation of the control member 308) causes the sliding assembly 324 coupled therewith to translate longitudinally as well. When the sliding assembly 324 is translated in the distal direction, a longitudinal distance along the major axis 218 between a connection point of the slider 326 with the hinge arm 320 and a connection point of the hinge arm 320 with the base 316 of the round blade 306 is reduced, and the hinge arm 320 pushes on the base 316 in a first non-normal direction relative to the major axis 218. In turn, the force of the hinge arm 320 upon the base 316 causes the proximal end 322 of the base 316 to translate toward a sidewall of the shaft 204, which rotates the round blade 306 about the fulcrum 314 (and thus, axis 370), thereby causing a “tilted” orientation or position of the round blade as shown in FIG. 3D. This orientation may correspond with the first position of the control member 308, as described above with reference to FIGS. 3A-3B. In the tilted orientation, a longitudinal axis of the round blade 306 (and/or a base 310 thereof) may be noncollinear or nonparallel with the major axis 218 of the shaft. For example, the longitudinal axis of the round blade 306 may be disposed at an angle of 45 degrees relative to the major axis 218 of the shaft 204.

When the sliding assembly 324 is translated in a proximal direction, a longitudinal distance along the major axis 218 between a connection point of the slider 326 with the hinge arm 320 and a connection point of the hinge arm 320 with the base 316 of the round blade 306 is increased, and the hinge arm 320 pulls on the base 316 in a first non-normal and proximal direction relative to the major axis 218. This, in turn, causes the proximal end 322 of the base 316 to translate proximally and toward a more central position within the shaft 204, which rotates the round blade 306 about the fulcrum 314 and causes a “straight” orientation or position of the round blade as shown in FIG. 3C. The straight orientation may correspond with the second position of the control member 308, as described above with reference to FIGS. 3A-3B. In the straight orientation, a longitudinal axis of the round blade 306 (and/or the base 310 thereof) may be collinear or parallel with the major axis 218 of the shaft.

The first position of the round blade 206, as shown in FIG. 3C, may be preferred for creating the horizontal incisions required for the creation of the tympanomeatal flap and for lifting the typanomeatal flap. The second position of the round blade 206, as shown in FIG. 3D, may be preferred for creating the two vertical incisions for the creation of the tympanomcatal flap.

FIGS. 3E-3G illustrate detailed cross-sectional side views of an exemplary locking mechanism 350 for a control member of a surgical knife, such as the surgical knife 300, according to embodiments described herein. In FIGS. 3E-3G, the control member 308 of surgical knife 300 is depicted for illustrative purposes. Generally, the control member 308 may be locked or maintained, via the locking mechanism 350, in at least a preset position A, wherein the round blade 306 operatively coupled to the control member 308 is disposed in the “straight” orientation, and a preset position B, wherein the round blade 306 is disposed in the “tilted” orientation. However, a locking mechanism 350 with three or more preset positions is also contemplated. Even further, in certain embodiments, the locking mechanism 350 may have continuous positioning, or be without fixed preset positions, such that the locking mechanism 350 may be locked or maintained at any point or increment along a range of positions. In such embodiments, the locking mechanism 350 may be secured in place by frictional forces between the control member 208 and the top of the channel 224 as caused by the biasing of the control member 208 by the spring 354.

Turning to FIG. 3E, the locking mechanism 350 comprises a spring-based locking mechanism having a spring 354 and one or more locking grooves 358 configured to mate with one or more protrusions 352 on the control member 308 (or the rod 312). Please note that other types of biasing members may also be used. In FIG. 3E, control member 308 is depicted as being held in the preset position A, where the control member 308 is positioned at a proximal end of the trough 356 and the operatively coupled to the connector 328. In this position, the spring 354, located at a bottom of the trough 356, presses the rod 312 against the top of the channel 224. This causes the one or more protrusions 352 of the control member 308, which are formed on an upper surface of the control member 308, to mate with the one or more locking grooves 358 formed on an upper surface of the channel 224, or, as shown in FIG. 3E, to abut an edge of the trough 356. This engagement secures the control member 308 and thus, the rod 312, in place. When in this position A, to laterally adjust the control member 308 and adjust the tilt angle of the round blade 306, a user must apply a downward force against the control member 308, which disengages the protrusion(s) 352 from the locking groove(s) 358 or trough 356, prior to laterally moving the control member 308.

In FIG. 3F, a downward force 360 is applied by the user to the control member 208, causing the spring 354 to flatten and the protrusion(s) 352 to disengage from the locking groove(s) 358 and/or the edge of the trough 356. The user then manually laterally translates control member 308 towards the distal end of the trough 356, causing the rod 312 and operatively to the connector to adjust the tilt of the round blade 306.

Turning now to FIG. 3G, upon translation of the control mechanism 308 towards the distal end of the trough 356, the one or more protrusions 352 reengage with the one or more locking grooves 358 and/or the trough 356, as facilitated by the upward biasing provided by the spring 354. Accordingly, the reengagement holds the control member 308 in the preset position B, wherein the round blade 306 is tilted downward. To readjust the control member 308 from the preset position B, the user may have to exert another downward force against the control member 308, and the spring 354 by association, to disengage the one or more protrusions 352 and enable lateral movement of the control member 308.

FIG. 4 illustrates a detailed perspective view of a round blade 406 of a surgical knife 400, according to embodiments described herein. The round blade 406 is generally representative of round blades 206 and 306 described above, and the surgical knife 400 is generally representative of surgical knives 200 and 300. For clarity, features of the surgical knife 400 like or similar to those of surgical knives 200 and 300 are labelled with like or similar reference numerals and are omitted from the following description for brevity.

As shown in FIG. 4, the round blade 406 couples to a shaft 404 of the surgical knife 400 at a distal end 414 of the shaft 404. In certain embodiments, the round blade 406 comprises a disc-like blade with a rounded cutting edge 410. For example, the round blade 406 may have a substantially circular or oval disc-like geometry with the rounded cutting edge 410. However, other morphologies are also contemplated, such as a substantially quadrilateral (or polygonal) geometry with the rounded cutting edge 410 on one (or more) sides. In certain embodiments, the round blade 406 may comprise both sharp (cutting) and dull (non-cutting) edges. For example, in embodiments where the round blade 406 comprises a circular, disc-like shape, only a most distal one-half to three-fourths or more, or one-fourth to three-fourths or more, of a circumference of the round blade 406 may be sharpened to form the cutting edge 410 for creating incisions. Meanwhile, the remainder of the circumference of the round blade 406 may be dull to prevent unwanted cutting or trauma to car tissues during use of the surgical knife 400.

FIG. 5 illustrates a schematic side view of another exemplary surgical knife 500, in an unrotated position as in FIG. 2A, according to certain embodiments herein. The surgical knife 500 is substantially similar to the surgical knife 200 and surgical knife 300, but further includes a suction tube 530 through which a vacuum pressure may be applied for clearing blood and/or debris during an otologic procedure and/or for increasing traction against otologic tissues being manipulated by the surgical knife 500. For clarity, features of the surgical knife 500 like or similar to those of surgical knives 200 and 300 are labelled with like or similar reference numerals and are omitted from the following description for brevity.

As shown, the suction tube 530 extends from a proximal portion of the surgical knife 500 to a distal portion of the surgical knife 500. More particularly, the suction tube 530 extends through the handle 202, through the shaft 204, and to the distal end 214 of the shaft 204. The suction tube 530 is fluidly coupled to a distal port 534 at the distal end 214 of the shaft 204, and further to a proximal port 532 formed in the handle 202. In certain embodiments, the proximal port 532 is formed in a proximal end 510 of the handle 202. The proximal port 532 generally comprises an opening configured to be fluidically coupled with a vacuum source, such as vacuum source of a surgical console, via flexible tubing (or other suitable connector) for supplying vacuum pressure to the suction tube 530 and thus, the distal port 534. Meanwhile, the distal port 534 comprises an opening disposed at any suitable location at the distal end 214 of the shaft 204 for applying the supplied vacuum pressure to external blood, debris and/or otologic tissues to “suck” them into or against the distal port 534. In certain embodiments, the distal port 534 is disposed through a sidewall of the shaft 204 below the round blade 206, or through the sidewall of the shaft 204 on a side of the round blade 206 opposite the channel 224.

As mentioned above, vacuum suction through the distal port 534 may be used during otologic procedures to clear blood, and/or debris from a surgeon's field of view, and/or to suction car tissues against the distal port 534 to facilitate easier manipulation thereof. For example, when the round blade 206 is utilized to push/lift the tympanomeatal flap during a postauricular middle ear surgery, a vacuum source fluidly coupled to the suction tube 530 may be activated to provide a vacuum pressure through the distal port 534 and aspirate/clear blood, debris, and secretions from the middle ear cavity, and/or the EAC, for better visualization of car structures by the surgeon. Alternatively, vacuum pressure may be provided through the distal port 534 to assist the surgeon in manipulating (e.g., lifting) the tympanomeatal flap. In such examples, the vacuum pressure generated by the vacuum source through proximal port 532 may be small enough to not damage the tympanomeatal flap and/or other ear tissues, but great enough to suction the tympanomeatal flap and elevate it with movement of the round blade 206.

In summary, embodiments of the present disclosure provide an otologic surgical knife having an adjustable round blade to facilitate the formation of different types of incisions with a single instrument, thereby reducing the number of instruments needed during certain otologic procedures. Such a surgical knife may be utilized, for example, to create and elevate/fold a tympanomeatal flap in the external auditory canal for access to the middle ear space. Because the surgical knife comprises an adjustable round blade, a surgeon may maintain and utilize the same knife to make various types of incisions in the external auditory canal. Thus, in certain examples, the surgical knife described herein reduces the number of instrument exchanges during an otologic procedure, which in turn increases the ease of use and convenience for the surgeon, improves procedural efficiency, and reduces the risk of trauma to the patient's ear tissues.

While the foregoing is directed to embodiments of the present disclosure, other and further embodiments of the disclosure may be devised without departing from the basic scope thereof, and the scope thereof is determined by the claims that follow.

### EXAMPLE EMBODIMENTS

Embodiment 1: A surgical knife, comprising: a handle comprising a distal end and a proximal end opposite the distal end, the handle further comprising a control member; a shaft coupled to and extending distally from the distal end of the handle, the shaft comprising a shaft axis; a rod disposed within the shaft and comprising a distal end and a proximal end opposite the distal end, wherein the proximal end of the rod is coupled to the control member of the handle and the distal end of the rod is disposed adjacent the distal end of the shaft; and a round blade movably coupled to the distal end of the rod, wherein actuation of the control member causes rotation of the round blade about an axis perpendicular to the shaft axis.
Embodiment 2: The surgical knife of Embodiment 1, wherein the rotation of the round blade about an axis perpendicular to the shaft axis causes the angle of the round blade to tilt relative to the shaft axis.
Embodiment 3: The surgical knife of Embodiment 1, wherein the round blade comprises a substantially circular or oval disc-like shape.
Embodiment 4: The surgical knife of Embodiment 1, further comprising a fulcrum mechanism coupling the round blade to the distal end of the rod to adjust a tilt angle of the round blade.
Embodiment 5: The surgical knife of Embodiment 1, wherein the control member comprises a sliding button, sliding toggle, a rotating knob, a leadscrew mechanism, a squeezing mechanism, or an electromotor controlled by a button or switch on the handle or another user input device.
Embodiment 6: The surgical knife of Embodiment 1, wherein the handle further comprises a locking mechanism for securing the control member in at least a first position and a second position.
Embodiment 7: The surgical knife of Embodiment 1, wherein the proximal end of the shaft is removably coupled to the handle.
Embodiment 8: The surgical knife of Embodiment 1, wherein the proximal end of the shaft is fixedly coupled to the handle.
Embodiment 9: The surgical knife of Embodiment 1, wherein the proximal end of the shaft is coupled to the handle at a non-zero angle relative to a major longitudinal axis of the handle.
Embodiment 10: The surgical knife of Embodiment 1, wherein the shaft is rotatable relative to the handle.
Embodiment 11: The surgical knife of Embodiment 10, wherein the proximal end of the shaft is coupled to the handle at a non-zero angle relative to a major longitudinal axis of the handle.
Embodiment 12: The surgical knife of Embodiment 1, wherein the handle further comprises a locking mechanism with continuous positioning for securing the control member at any position along a range of positions.

What is claimed is:

1. A surgical knife, comprising:
a handle comprising a distal end and a proximal end opposite the distal end, the handle further comprising a control member;
a shaft comprising a distal end and a proximal end opposite the distal end, the proximal end coupled to the distal end of the handle;
a rod disposed within the shaft and comprising a distal end and a proximal end opposite the distal end, wherein the proximal end of the rod is coupled to the control member, and wherein the rod is configured to rotate about a first rotation axis upon actuation of the control member; and
a round blade rotatably coupled to the distal end of the rod and configured to rotate about a second rotation axis, wherein the second rotation axis is disposed at a fixed, non-zero angle relative to the first rotation axis.

2. The surgical knife of claim 1, wherein the round blade comprises a substantially circular or oval disc-like shape.

3. The surgical knife of claim 2, wherein only a portion of a circumference of the round blade comprises a sharpened cutting edge.

4. The surgical knife of claim 1, wherein the second rotation axis is between 0 degrees and 90 degrees relative to the first rotation axis.

5. The surgical knife of claim 1, wherein the round blade rotates about the second rotation axis at a rotational angle between 0 degrees and 360 degrees.

6. The surgical knife of claim 1, wherein the round blade is coupled to the distal end of the rod by a universal joint or bevel gears.

7. The surgical knife of claim 1, wherein the control member comprises a rotating component, a sliding button driving a leadscrew, a squeezing mechanism, or an electromotor controlled by a button.

8. The surgical knife of claim 1, wherein the handle further comprises a locking mechanism for securing the control member in at least a first position and a second position.

9. The surgical knife of claim 1, further comprising:
a suction tube disposed within the shaft, the suction tube in fluid communication with a distal port disposed at the distal end of the shaft and a proximal port disposed in the handle, wherein the proximal port is configured to be operably coupled with a vacuum source to provide a vacuum pressure at the distal port.

10. The surgical knife of claim 1, wherein the proximal end of the shaft is removably or fixedly coupled to the handle.

11. The surgical knife of claim 1, wherein the proximal end of the shaft is coupled to the handle at a non-zero angle relative to a major longitudinal axis of the handle.

12. The surgical knife of claim 1, wherein the shaft is rotatable relative to the handle.

13. The surgical knife of claim 12, wherein the proximal end of the shaft is coupled to the handle at a non-zero angle relative to a major longitudinal axis of the handle.

14. The surgical knife of claim 8, wherein the handle further comprises a locking mechanism with continuous positioning for securing the control member at any position along a range of positions.

15. The surgical knife of claim 1, wherein the shaft further comprises a fulcrum mechanism for allowing adjustment of a tilt angle of the round blade.
